(19) United States
(12) Patent Application Publication
(10) Pub. No.: US 2003/0059932 A1
(43) Pub. Date: Mar. 27, 2003

(54) Photobioreactor

(75) Inventors: James S Craigie, Halifax (CA); Stephen M. Armstrong, Halifax (CA); Larry S. Staples, Halifax (CA); Andrew G. Bauder, Ottawa (CA)

Correspondence Address: OGILVY RENAULT, 1981 MCGILL COLLEGE AVENUE, SUITE 1600, MONTREAL, QC, H3A2Y3, CA

(73) Assignee: National Research Council of Canada

(21) Appl. No.: 10/200,527

(22) Filed: Jul. 23, 2002

Related U.S. Application Data
(60) Provisional application No. 60/306,899, filed on Jul. 23, 2001.

(51) Int. Cl. C12M 1/00

(52) U.S. Cl. 435/292.1; 47/1.4

(57) ABSTRACT

A photobioreactor for mass production of algae in a liquid pool, comprising a vessel including first and second generally parallel walls. The vessel is adapted to receive a liquid pool. A plurality of hollow tubes extends from the first wall to the second wall for receiving a light source. The hollow tubes are adapted to be immersed in the liquid pool such that the light source can illuminate the liquid pool. The hollow tubes are accessible from outside of the vessel for allowing for the servicing of the light source without having to shut down operation of the photobioreactor. Inlet ports are provided for injecting fluids into the vessel. Outlet ports are provided for extracting liquid from the vessel.

### CROSS-REFERENCE

This application claims priority on U.S. Provisional Patent Application No. 60/306,899 filed Jul. 23, 2001.

### BACKGROUND OF THE INVENTION

1. Field of the Invention

The present invention relates to the production of microalgae (or phytoplankton) and, more particularly, to a photobioreactor for the mass production of microalgae.

2. Description of the Prior Art

Production of microalgae is required for a variety of applications. In aquaculture, selected species of microalgae with desirable nutritional profiles are cultured as food for broodstock, larvae, and juvenile shellfish. They are also used to enhance the nutritional characteristics of zooplankton such as rotifers cultured in finfish hatcheries as food for early stage larvae. Large volumes of microalgae have to be produced indoors in temperature-controlled areas to meet the requirements of such hatcheries generally during times unfavorable for algal production using natural light. Moreover, algal production must be reliable to meet daily requirements and sustainable for long periods. Because the microalgae are used as feed, the cultures must be kept free of potential pathogens and opportunistic algae Certain forms of zooplankton that graze heavily on microalgae must also be excluded from the system in order to sustain acceptable yields and quality of algae.

In most hatcheries, microalgae are produced indoors in large, upright, transparent vessels, usually polyethylene bags or self-standing fiberglass cylinders. These are commonly illuminated by an external bank of fluorescent lamps. Although some cultures are grown in greenhouses, the usual practice is to house them in a single room that may have provision, but often does not, for air conditioning.

Numerous photobioreactor designs for the culture of microalgae, described in the scientific and patent literature, are open to the environment and are outdoors. In temperate zone countries such as Canada, where cold-water aquaculture is practiced, shellfish hatcheries begin their rearing operations in the autumn and winter seasons, quite out of synchrony with the light and temperature regimes most favorable for outdoor mass production of algae. The spawning cycle of several commercially important marine fish that are artificially propagated also occurs during winter and early spring, and hatchery operators must be able to access quality feed to rear the larvae. Therefore, large tank systems, open raceways and outdoor ponds for commercial production of microalgae are used with a limited number of species in locations where environmental conditions permit.

The production of microalgal biomass in hatcheries is labor intensive and occupies considerable space because most cultivation systems now in use produce algae at relatively low cell densities. Open cultures frequently become contaminated with undesirable bacteria and other organisms and therefore become unsuitable as feed, as opposed to closed systems, which prevent these failures due to contamination from opportunistic organisms. Most of the simple systems have little or no provision for temperature and pH control during operation, leading to sub-optimum algal growth performance and, all too frequently, catastrophic loss of cultures. Individual needs of microalgal species produced in a particular facility cannot be addressed because the culture vessels are usually in a common room under one selected set of conditions. These systems frequently require a significant amount of floor space and are clumsy and laborious to operate and clean. Some of these may operate well, but their capital and operational costs prohibit their use from many applications, such as aquaculture.

There are photobioreactors on the market that address the various issues described above but these are too expensive to be used in all but the largest hatchery operations Production of live microalgae in such hatcheries is done under artificial light in temperature controlled rooms maintained at each hatchery. Furthermore, they frequently are relatively complex to operate. Such systems limit flexibility in the type and number of species that could be produced in an aquaculture facility. Existing systems also make relatively inefficient use of light energy, which significantly increases their operating costs.

U.S. Pat. No. 5,104,803 issued on Apr. 14, 1992 to Delente discloses a photobioreactor in which light banks are mounted side by side in a tank containing a liquid culture, The banks are positioned in the tank so that the light emitting surfaces thereof are substantially totally immersed in the liquid. Each of the lighting units is made up of a plurality of light tubes disposed in close proximity to one another with their longitudinal axis lying generally in the same plane. Also, the light banks each include an enclosure for the electrical leads and end portions of light tubes to render these portions impervious to the liquid culture when immersed in the culture, so that the light emitted by these end portions is not transmitted to the liquid culture. Electrical leads are connected to the electrical contacts of the light tubes and extend from the light bank to allow connection to external electrical power source, The entire light emitting structure of the light bank can thus be immersed In the liquid culture.

U.S. Pat. No. 5,162,051 issued on Nov. 10, 1992 to Hoekeema is presented as an improvement over U.S. Pat. No. 5,104,803. Namely, problems have resulted from photobioreactor designs such as the one described in U.S. Pat. No. 5,104,803, which have utilized light banks and light compartments immersed in the liquid culture. Firstly, it is difficult to safely and effectively make the necessary electrical connections with the light tubes. Secondly, access to the light tubes for maintenance is made more difficult. Consequently, U.S. Pat. No. 5,162,051 introduces light transmitting baffles mounted side by side in a tank containing a liquid culture. Each baffle defines a hollow cavity within planar walls and is mounted so that the cavity is accessible from outside of the tank for the insertion of a light source therein, The sides of the baffles are constructed of optically transparent material to allow the light from the light source to be transmitted to the liquid which is in contact with the outside surfaces of the baffles. Each light source is made up of a plurality of light tubes supported by braces or similar supporting structures and mounted in the baffles. Electrical leads are extended from the tubes to allow connection with an external power source.

A few design factors are involved in reproducing an adequate environment for the production of algae. An important design factor resides in exposing the entire algal culture to an optimal amount of light. The light exposure is critical as algae are sensitive to the amount and kind of light. Light of excessive intensity may be harmful to algae, while insufficient light will result in low levels of photosynthesis. Furthermore, productivity of algal cells is known to respond positively when the cells are exposed to fluctuating levels of light. The ability to control the photoperiod is an issue as continuous light may be deleterious for certain fastidious phytoplankton species.

Heat is another important parameter in the design for optimal algal production. The production of algae is most efficient within predetermined ranges of temperatures, which, in turn, are species dependent. Consequently, means must often be provided for independently controlling the temperature of the algal culture. Also, pH control is a critical parameter to consider during the design of a photobioreactor. This is achieved by on-demand delivery of carbon dioxide, a key metabolic substrate, at rates commensurate with growth of the algae. The ideal pH range for a given alga may be narrow; however, this range can vary from species to species.

### SUMMARY OF THE INVENTION

It is therefore an aim of the present invention to provide a closed system photobioreactor adapted to produce over long periods substantially pathogen-free microalgae of consistent quality at high cell densities.

Therefore, in accordance with the present invention, there is provided a photobioreactor for mass production of microalgae in a liquid pool, comprising: a vessel including at least first and second generally parallel walls, and being adapted to receive a liquid pool, at least one hollow tube extending from said first wall to said second wall, for receiving a light source, said hollow tube being adapted to be immersed in the liquid pool such that the light source can illuminate the liquid pool, said hollow tube being accessible from outside of said vessel for allowing for the servicing of the light source without having to shut down operation of said photobioreactor, at least one inlet port for injecting at least one fluid in said vessel, and at least one outlet port for extracting a liquid from said vessel.

### BRIEF DESCRIPTION OF THE DRAWINGS

Having thus generally described the nature of the invention, reference will now be made to the accompanying drawings, showing by way of illustration a preferred embodiment thereof, and in which:

FIG. 1 is an exploded perspective view of a photobioreactor in accordance with the present invention;

FIG. 2 is an exploded perspective view of a flange seal assembly of the photobioreactor of the present invention;

FIG. 3 is a perspective view of an air sparger of the photobioreactor of the present invention;

FIG. 4 is a perspective view of a cooling coil of the photobioreactor of the present invention; and

FIG. 5 is an exploded perspective view of a viewport of the photobioreactor of the present invention.

### DESCRIPTION OF THE PREFERRED EMBODIMENTS

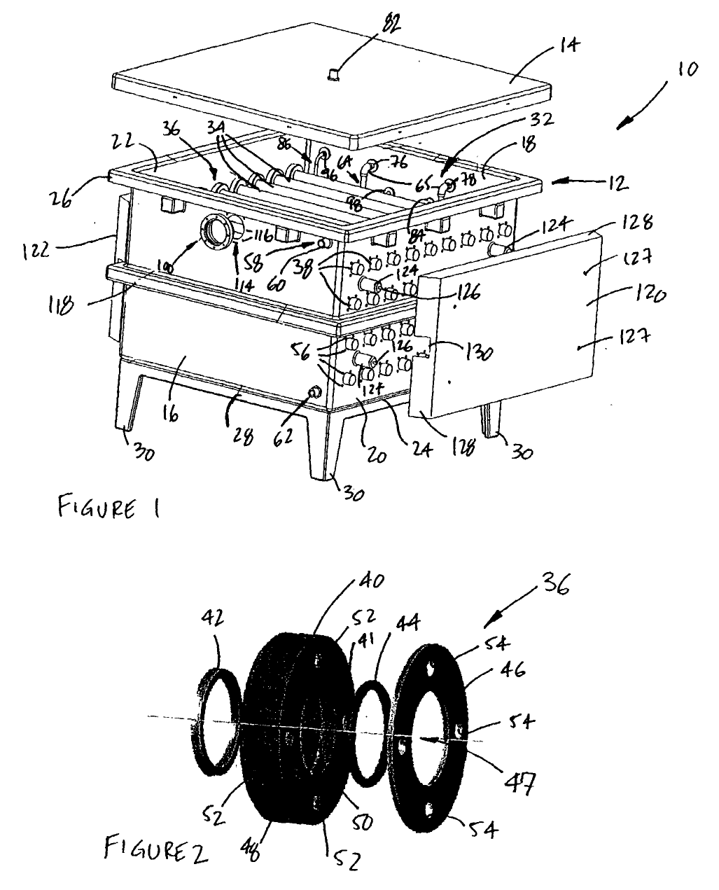

Referring now to the drawings, a photobioreactor is generally shown at 10 in FIG. 1. The photobioreactor 10 comprises a tank 12 and a cover 14. The tank 12 is defined by a front wall 16, a back wall 18, lateral walls 20 and 22 and a bottom wall 24. A ledge 26, outwardly projecting at the top of the front and back walls 16 and 18 and of the lateral walls 20 and 22, co-acts with the cover 14 to seal the tank 12. Draw latches (not shown) may be used to facilitate the releasable locking of the cover 14 to the tank 12. The tank 12 sits on a stand 28 comprising legs 30. The tank 12 may be of any convenient shape, but for the embodiment described herein, a, generally rectangular shape is preferred. It is pointed out that the photobioreactor 10 of the present invention is preferably provided with the cover 14. Although not necessary, the cover 14 ensures that the liquid pool in the photobioreactor 10 is not open to the environment, thereby substantially reducing the risk that it becomes contaminated.

The photobioreactor 10 defines a vessel, generally shown at 32 in FIG. 1. The vessel 32 contains a liquid pool for the culture of algae. The vessel 32 comprises a bank of sleeves 34, extending between the opposed lateral walls 20 and 22. The number and the disposition of the sleeves 34 are chosen in accordance with the volume of the photobioreactor. The sleeves 34 further extend through the lateral walls 20 and 22. Light emitting sources (not shown) can thus be inserted in the sleeves 34 from the outside of the tank. 12. Light emitting sources are known in the art, such as fluorescent tubes or the like.

The sleeves 34 are supported at opposed ends thereof within apertures 38 defined in the lateral walls 20 and 22. The sleeves 34 and apertures 38 are hermetically sealed by flange seal assemblies 36. One of the flange seal assemblies 36 is shown in more detail in FIG. 2. The flange seal 36 comprises an annular flange 40, a lip ring 42, an O-ring 44 and an annular gasket 46. The annular flange 40 defines an opening 41. The opening 41 comprises recesses 48 and 50 for receiving the lip ring 42 and the O-ring 44, respectively. The annular gasket 46, defining an opening 47, is sandwiched between the annular flange 40 and the inner surface of lateral walls 20 or 22. The annular flange 40 further comprises tapped holes 52, equidistantly spaced thereon. The tapped holes 52 are aligned with holes 54 in the annular gasket 46 and with holes 56 defined around the apertures 38 of the lateral walls 20 and 22. Similarly, the openings 41 and 47 of the annular flanges 40 and the annular gaskets 46, respectively, are aligned with apertures 38 of the lateral walls 20 and 22, thereby forming holes for the insertion of the sleeves 34 therethrough. The annular flanges 40 may thereby be bolted to the lateral walls 20 and 22. The sleeves 34 are inserted in the flange seal assembly and co-act with the lip ring 42 and O-ring 44 to provide a sealed connection. Furthermore, the annular gaskets 46 seal the annular flanges 40 to the lateral walls 20 and 22.

As seen in FIG. 1, the photobioreactor 10 further comprises an inlet port 58. The inlet port SR is located at a top end corner of the front wall 16 of the tank 12 and extends therethrough. An outer end 60 of the inlet port 58 is adapted to be connected to control valves, piping or other. similarly, an outlet port (not shown) is located at a corner of the bottom wall 24. The outlet port is adapted to be connected to valves, in order to close the outlet and control the discharge of the photobioreactor 10. A sampling port 62 is located at a bottom end corner of the front wall 16 and is adapted to be connected to a valve to control the sampling of the tank 12. The inlet port 58, the outlet port and the sampling port 62 are each sealed co walls of the tank 12 by a sealing assembly, such as the flange seal assembly 36. It is observed that the above-described ports may be positioned on any wall defining the tank 12. For instance, the inlet port 58 may be provided in the cover 14.

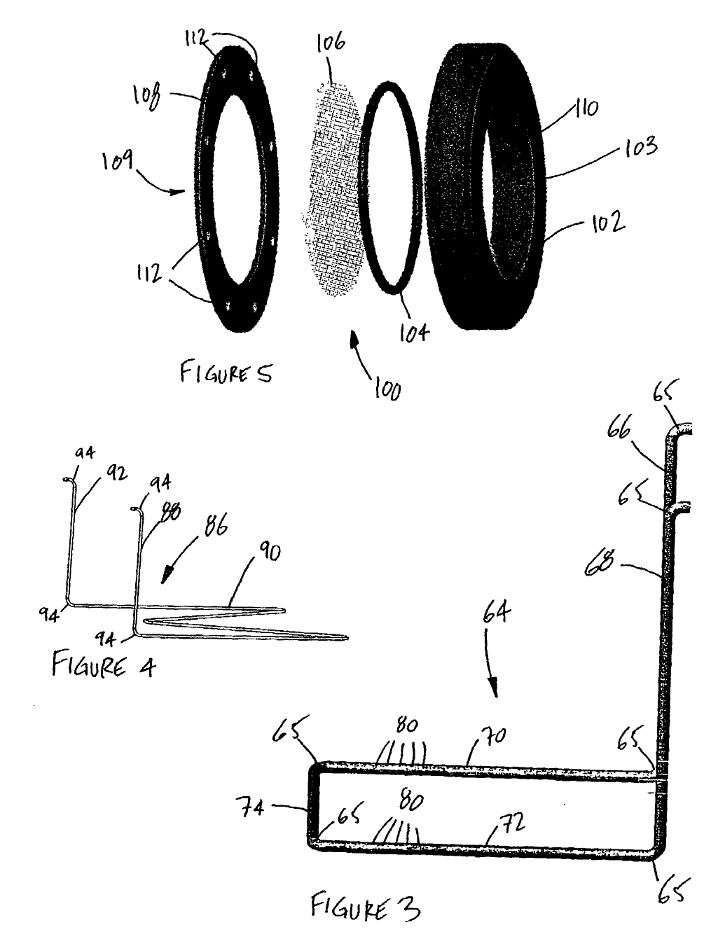

The photobioreactor 10 comprises a sparger 64. The sparger 64 is best shown in FIG. 3. The sparger 64 is defined by parallel vertical pipes 66 and 68, connected to horizontal pipes 70 and 72, respectively, by elbow connectors 65, as known in the art. The horizontal pipes 70 and 72 are joined by elbow connectors 65 to a horizontal pipe 74. The vertical pipes 66 and 68 of the sparger 64 extend along the back wall 18 of the tank. Similarly, the horizontal pipes 70, 72 and 74 extend along adjacent the bottom wall 24. Top ends of the vertical pipes 66 and 68 also comprise elbow connectors 65 that are connected to ports 76 and 78, at a top end of the back wall 18 The ports 76 and 78 are sealed to the back wall 18 by a sealing assembly, such as the flange seal assembly 36. A plurality of pin holes 80 are spread apart on the horizontal pipes 70 and 72. Sources of pressurized air and carbon dioxide are connected to both ports 76 and 78, thereby injecting the gases in the photobioreactor 10 through the plurality of pin holes 80 on the horizontal pipes 70 and 72. Sparging with air provides a significant amount of the carbon dioxide consumed as required in photosynthesis and effectively removes excess oxygen generated by the algae. In this way, the algae are protected from damage due to excess oxygen supersaturation and associated photo-oxidative processes. Furthermore, the injection of gases at the bottom of the photobioreactor 10 results in the mixing in the liquid pool. The effective mixing also facilitates good temperature and pH regulation of the culture.

An air vent valve 82 (FIG. 1), as known in the art, is located on top of the cover 14, and may be connected to an exhaust manifold to allow the used air to be vented outdoors. Furthermore, the exhaust manifold allows the release of excess pressure in the photobioreactor 10 resulting from the injection of air and carbon dioxide through the sparger 64.

A pH controller, also known in the art, monitors the pH of the liquid pool by a pH sensor located at 84 on the back wall 18. The pH controller ensures that the pH of the liquid pool remains within a predetermined range. This is done by the pH controller modulating the input of carbon dioxide in order to adjust the pH of the liquid pool with precision (e.g. ±0.1 unit of pH).

A cooling coil 86 is best shown in FIG. 4. The cooling coil 86 comprises an inlet vertical portion 88, a coil portion 90 and an outlet vertical portion 92. These portions 88, 90 and 92 are connected together by elbow connectors 94. Further elbow connections 94 are provided at the top ends of the inlet vertical portion 88 and of the outlet vertical portion 92 The inlet vertical portion 86 and the outlet vertical portion 92 extend along the inner surface of the back wall 10 of the tank 12. The elbows 94 provided at the top ends of the inlet and outlet vertical portions 88 and 92 are connected to an inlet port 96 and an outlet port (not shown) disposed through the back wall 18. The inlet 96 and outlet ports are sealed to the back wall 18 by a sealing assembly, such as the flange seal assembly 36 of FIG. 2. The cooling coil 86 is wall-mounted as opposed to being positioned on the bottom wall of the tank to facilitate the cleaning and prevent the deposition of algae.

A cooling fluid is injected in the cooling coil 86 by the inlet port 96 and circulates in succession through the inlet vertical portion 88, the coiled portion 90 and the outlet vertical portion 92 to then exit through the outlet port. As the cooling coil 86 is in the liquid pool of the vessel 32, the cooling fluid absorbs liquid pool heat as it circulates within the cooling coil 86. The cooling coil 86 is thus made of materials enhancing heat transfer. A temperature controller, also known in the art, monitors the temperature of the liquid pool by a temperature probe located at 98 on the back wall 18 at the tank 12. The temperature controller modulates the flow of cooling fluid through the cooling coil 86 to ensure that the temperature of the liquid pool remains within the predetermined temperature range. As for heating, the liquid pool absorbs heat emitted by the light source.

A viewport is generally shown at 100 on the tank 12 in FIG. 1. As seen in FIG. 5, the viewport 100 comprises an annular flange 102, an O-ring 104, a sight glass 106 and an annular gasket 108. The annular flange 102 defines a sight hole 103 and a counterbore 110. The annular flange 102 further comprises tapped holes (not shown), equidistantly located thereon. The sight glass 106 is inserted in the counterbore 110, thereby sandwiching the O-ring 104 to the counterbore 110. The sight hole 103 of the annular flange 102 is aligned with a sight hole 109 defined by the annular gasket 108 and with a sight hole 114 defined in the front wall 16. The tapped holes on the annular flange 102 are aligned with holes 112 defined in the annular gasket 108 and holes 116 defined in the front wall 16. The annular flange 102 can thus be bolted to the front wall 16. The annular gasket 109 is sandwiched between the annular flange 102 and the front wall 16, thereby providing a seal therebetween. As mentioned above, the O-ring 104 seals the annular flange 102 from the sight glass 106, thereby hermetically connecting the viewport 100 to the front wall 16.

Now referring to FIG. 1, a rib 118 horizontally surrounds the outer perimeter of the tank 12 and serves to structurally strengthen the tank. The rib 118 is generally located in the middle of the front wall 16, the back wall 18 and the lateral walls 20 and 22 Side guards panels 120 and 122 are used for protecting the electrical wiring and connections of the light emitting sources within the sleeves. The side guard panels 120 and 122 define holes 127 that are engaged by threaded pins 126 located at ends of rods 124. The side guard panels 120 and 122 are secured between the rods 124 and nuts (not shown) threadably engaged on the threaded pins 126 on the outside of the side guard panels 120 and 122. The side guard panels 120 and 122 further comprise peripheral flanks 128, Lateral ones of the flanks 128 of the side guard panels 120 and 122 define grooves 130, co-acting with the rib 118 for bringing additional support to the side guard panels 120 and 122.

The photobioreactor 10 described in the present invention will serve mainly, but not exclusively, for the production of (1) microalgae for feeding shellfish in aquaculture hatcheries, (2) microalgae for feeding rotiters and Artemia destined to become live feed for early stage fish larvae in hatcheries, (3) microalgae for greening water in larval fish rearing facilities, (4) algal biomass for use as neutraceuticals, feed ingredients or health foods, and (5) algal biomass for extraction of valuable compounds. An interesting feature of the photobioreactor resides in the fact that it is a hermetically closed system with controlled inlets and outlets. Such control is beneficial in providing ideal conditions within the closed system. For example, filters are used upstream of the air sparger 64 to ensure that the air and carbon dioxide injected are sterilized. Easy access to the vessel 34 through the cover 14 allows for the interior surface of the photobioreactor 10 to be chemically sterilized, using hypochlorite or comparable solutions.

The important parameters such as pH, temperature, irradiance levels, photoperiod, nutrient input and output, as well as high quality treated water (sterilized or pasteurized), seawater or freshwater, will be added or controlled automatically, and this is easily achieved by the design of the photobioreactor 10 of the present invention, whereby numerous inlets and outlets may be provided with the photobioreactor 10 for the injection of desired fluids. These inlets and outlets may be fully automated, and along with the pH and temperature controllers described above, provide for a consistent quantity and quality of the liquid culture output. A system of valves may be used in relation with the various elements of the system. For example, a solenoid valve controls the flow rate of cooling fluid through the cooling coil 86, thereby enabling the liquid pool to remain within a predetermined range of temperature (e.g. precision of 0.5° C.). Such control automation may similarly be provided for the inlet port 58, the outlet port and the air sparger 64. Furthermore, the photobioreactor 10 of the present invention may be operated in semi-continuous or continuous operation for periods of several weeks, wherein periodic or constant outflow of algae from the photobioreactor is compensated by a generally equivalent inflow of sterile nutrient solutions and water. Consequently, the specific design of the photobioreactor 10 and the strategic positioning of the ports provide the ability to harvest based on pre-set cell biomass as opposed to standard overflow rate, with the positioning of the outlet port (not shown) at the bottom of the photobioreactor.

The construction of the photobioreactor 10 is sufficiently rugged to withstand the weight and the pressure of the liquid pool inside. The lifetime of the vessel 32 is expected to be indefinitely long. By its specific design, the photobioreactor 10 can be scaled up to larger sizes, and it is economical to construct and readily serviced. The photobioreactor 10 requires minimum space, allowing the use of many photobioreactors in hatcheries. Thus, various types of microalgae may be produced at the same time in a hatchery. In another interesting feature, the photobioreactor 10 may be coated on its exterior with foam insulation for use under cold ambient conditions.

As described above, illumination is provided internally by fluorescent lamps individually housed in the transparent sleeves 34 passing through the culture, and this illumination is more efficient than in prior art devices as the culture liquid surrounds each fluorescent lamp (as the latter is lodged in its own cylindrical sleeve 34). Indeed, as the sleeves 34 are totally immersed in the liquid pool within the closed vessel 32, virtually all the emitted light is absorbed by the algal culture. Furthermore, the strategic positioning of the sleeves 34 ensures that light is well distributed throughout the liquid pool. The advantage of fluorescent tubes is that light is efficiently emitted in a generally uniform manner along the length of the tube and is perpendicular in all directions.

The use of suitable ballasts in series with the fluorescent lamps allows for a consistent intensity of light. The sleeves 34 may be made of glass or other suitable clear, transparent tubing. The multiple light sources are geometrically arranged so as to illuminate the maximum portion of the culture volume, yet remain compatible with other operational demands of the system. Also, because the ends of the sleeves 34 are open, the electrical wiring of the tubes is easily and safely laid. For these reasons, replacement of a fluorescent tube can be made during algal culture without jeopardizing the quality and longevity of the output. Individual cells suspended in the fluid medium by the air sparger-induced mixing are thereby propelled through regions, of relatively high and low light between and amongst the fluorescent lamps of the photobioreactor 10. The productivity of algal cells will be enhanced by the fluctuating levels of light.

We claim:

1. A photobioreactor for mass production of microalgae in a liquid pool, comprising: a vessel including at least first and second generally parallel walls, and being adapted to receive a liquid pool, at least one hollow tube extending from said first wall to said second wall, for receiving a light source, said hollow tube being adapted to be immersed in the liquid pool such that the light source can illuminate the liquid pool, said hollow tube being accessible from outside of said vessel for allowing for the servicing of the light source without having to shut down operation of said photobioreactor, at least one inlet port for injecting at least one fluid in said vessel, and at least one outlet port for extracting a liquid from said vessel.

2. The photobioreactor according to claim 1, further comprising a control system for controlling temperature of the liquid pool.

3. The photobioreactor according to claim 1, further comprising a control system for controlling the pH of the liquid pool.

4. The photobioreactor according to claim 1, wherein a protective cover is provided outwardly of at least one of said first and second walls of said vessel for selectively covering an access end of said hollow tube and wiring associated with the light source.

5. The photobioreactor according to claim 1, further comprising a control system for controlling the injection of the fluid through said at least one inlet port and the extraction of the liquid through said at least one outlet port.

6. The photobioreactor according to claim 1, wherein a lid is provided for selectively hermetically enclose the liquid pool in said vessel of said photobioreactor.

7. The photobioreactor according to claim 1, wherein a wall of said vessel has a sight glass extending therethrough.

8. The photobioreactor according to claim 1, wherein production can be achieved over long periods of substantially pathogen-free microalgae of consistent quality of high cell densities.

9. The photobioreactor according to claim 1, wherein there are provided a series of hollow tubes extending in a substantially parallel way from said first wall to said second wall.

10. The photobioreactor according to claim 9, wherein a protective cover is provided outwardly of at least one of said first and second walls of said vessel for selectively covering access ends of said hollow tubes and wiring associated with the light source.

11. The photobioreactor according to claim 1, wherein said hollow tube extends through at least one of said first and second walls.

12. The photobioreactor according to claim 11, wherein there are provided a series of hollow tubes extending in a substantially parallel way from said first wall to said second wall and through at least one of said first and second walls.

13. The photobioreactor according to claim 12, wherein a protective cover is provided outwardly of at least one of said first and second walls of said vessel for selectively covering access ends of said hollow tubes and wiring associated with the light source.
